(19) United States
(12) Patent Application Publication
(10) Pub. No.: US 2022/0362516 A1
(43) Pub. Date: Nov. 17, 2022

(54) Catheter Assembly and Related Devices and Methods

(71) Applicant: Becton, Dickinson and Company, Franklin Lakes, NJ (US)

(72) Inventors: John Lackey, West Valley City, UT (US); Megan Scherich, Salt Lake City, UT (US); Jonathan Karl Burkholz, Salt Lake City, UT (US); Weston F. Harding, Lehi, UT (US); Ralph L. Sonderegger, Farmington, UT (US); Curtis H. Blanchard, Riverton, UT (US); Hadley Hendrick, Los Angeles, CA (US)

(21) Appl. No.: 17/743,941

(22) Filed: May 13, 2022

Related U.S. Application Data
(60) Provisional application No. 63/188,821, filed on May 14, 2021.

(51) Int. Cl. A61M 25/00; A61M 39/10

(52) U.S. Cl. CPC A61M 25/0097; A61M 39/10; A61M 2039/1077; A61M 2039/0036

(57) ABSTRACT

A catheter assembly may include a catheter adapter, which may include a distal end, a proximal end, an inner surface extending through the distal end and the proximal end and forming a lumen, and a side port forming a side port pathway through a sidewall of the catheter adapter and in fluid communication with the lumen. The catheter assembly may include a catheter extending distally from the distal end of the catheter adapter. The catheter assembly may include a septum disposed within the lumen proximal to the side port pathway. A portion of the inner surface proximal to the catheter and distal to the septum may include one or more of the following: a textured surface, a channel configured to direct fluid flowing into the lumen from the side port, a protrusion extending inwardly into the lumen opposite the side port pathway, and an annular shoulder.

### CROSS-REFERENCE TO RELATED APPLICATION

The present application claims priority to U.S. Provisional Application Ser. No. 63/188,821, entitled “Catheter Assembly and Related Devices and Methods”, filed May 14, 2021, the entire disclosure of which is hereby incorporated by reference in its entirety.

### BACKGROUND OF THE INVENTION

A catheter is commonly used to infuse fluids into vasculature of a patient. For example, the catheter may be used for infusing normal saline solution, various medicaments, or total parenteral nutrition. The catheter may also be used for withdrawing blood from the patient.

The catheter may include an over-the-needle peripheral intravenous (“IV”) catheter. In this case, the catheter may be mounted over an introducer needle having a sharp distal tip. The catheter and the introducer needle may be assembled so that the distal tip of the introducer needle extends beyond the distal tip of the catheter with the bevel of the needle facing up away from skin of the patient. The catheter and introducer needle are generally inserted at a shallow angle through the skin into vasculature of the patient.

In order to verify proper placement of the introducer needle and/or the catheter in the blood vessel, a clinician generally confirms that there is “flashback” of blood in a flashback chamber of a catheter assembly that includes the catheter. After placement of the needle has been confirmed, the clinician may remove the needle, leaving the catheter in place for future blood withdrawal or fluid infusion.

Many catheter assemblies have a septum proximal to an extension tube inlet, creating a region distal to the septum that is difficult to flush free of fluids (such as blood or infusates). Stagnant fluid within the region distal to the septum may lead to accumulation of bacteria within the catheter assembly, which may result in infection or removal of the catheter from the patient.

The subject matter claimed herein is not limited to embodiments that solve any disadvantages or that operate only in environments such as those described above. Rather, this background is only provided to illustrate one example technology area where some implementations described herein may be practiced.

### SUMMARY OF THE INVENTION

The present disclosure relates generally to vascular access devices, systems, and methods. More particularly, the present disclosure relates to a catheter assembly, as well as related devices and methods. In some embodiments, the catheter assembly may facilitate flushing of the catheter assembly to avoid stagnant fluid within the catheter assembly. For example, in some embodiments, the catheter assembly may include one or more features to induce turbulent flow within the catheter assembly. As another example, in some embodiments, the catheter assembly may decrease a volume and/or change a geometry of a region within the catheter assembly that may otherwise be difficult to flush.

In some embodiments, the catheter assembly may include a catheter adapter, which may include a distal end, a proximal end, and an inner surface extending through the distal end and the proximal end. In some embodiments, the inner surface of the catheter adapter may form a lumen. In some embodiments, the catheter adapter may include a side port forming a side port pathway through a sidewall of the catheter adapter and in fluid communication with the lumen. In some embodiments, the catheter assembly may include a catheter extending distally from the distal end of the catheter adapter. In some embodiments, the catheter assembly may include a septum, which may be disposed within the lumen proximal to the side port pathway.

In some embodiments, a portion of the inner surface proximal to the catheter and distal to the septum may include a textured surface. In some embodiments, the textured surface may be rough. In some embodiments, the textured surface may include multiple depressions or protrusions. In some embodiments, each of the depressions or protrusions may be dome shaped. In some embodiments, the depressions or protrusions may be arranged in a pattern. In some embodiments, the textured surface may extend into the side port pathway.

In some embodiments, the portion of the inner surface proximal to the catheter and distal to the septum may include a channel configured to direct fluid flowing into the lumen from the side port. In some embodiments, the portion of the inner surface may be molded within a threaded core pin. In some embodiments, the channel may include a thread. In some embodiments, the channel intersects the side port pathway. In some embodiments, the channel may be disposed distal to the side port pathway. In some embodiments, fluid flowing into the lumen from the side port is configured to enter a first end of the channel and flow towards a second end of the channel. In some embodiments, the second end of the channel may be disposed towards the side port compared to the first end of the channel.

In some embodiments, the portion of the inner surface proximal to the catheter and distal to the septum may include an annular shoulder. In some embodiments, the annular shoulder may be disposed at a 90-degree angle. In some embodiments, the catheter assembly may include the wedge securing the catheter within the distal end of the catheter adapter, and the shoulder may be proximate a proximal end of the wedge. In some embodiments, a distal end of the septum may be aligned with or distal to the side port pathway.

In some embodiments, the catheter assembly may include an insert disposed within the catheter adapter. In some embodiments, the insert may extend across the lumen and selectively seal the lumen. In some embodiments, the insert may include a two-way valve disposed distal to the side port pathway. In some embodiments, the insert may include a slit or leaflets. In some embodiments, the insert may be configured to remain closed unless a pressure differential exceeds a predetermined pressure differential.

In some embodiments, the catheter assembly may include the septum disposed within the lumen, and a distal end of the septum may include an arm extending distally and contacting the inner surface. In some embodiments, the arm may be on an opposite side of the lumen as the side port. In some embodiments, a distal end of the arm may include an extension extending inwardly.

It is to be understood that both the foregoing general description and the following detailed description are examples and explanatory and are not restrictive of the invention, as claimed. It should be understood that the various embodiments are not limited to the arrangements and instrumentality illustrated in the drawings. It should also be understood that the embodiments may be combined, or that other embodiments may be utilized and that structural changes, unless so claimed, may be made without departing from the scope of the various embodiments of the present invention. The following detailed description is, therefore, not to be taken in a limiting sense.

### BRIEF DESCRIPTION OF THE DRAWINGS

Example embodiments will be described and explained with additional specificity and detail through the use of the accompanying drawings in which:

FIG. 1 is a cross-sectional view of a prior art catheter assembly;

FIG. 2A is a cross-sectional view of an example catheter assembly, illustrating an example textured surface, according to some embodiments;

FIG. 2B is a cross-sectional view of an example feature, according to some embodiments;

FIG. 2C is a cross-sectional view of another example feature, according to some embodiments;

FIG. 3A is a cross-sectional view of the catheter assembly, illustrating an example channel, according to some embodiments;

FIG. 3B is an upper perspective view of the catheter assembly, illustrating the channel, according to some embodiments;

FIG. 4 is a cross-sectional view of the catheter assembly, illustrating another example channel, according to some embodiments;

FIG. 5 is a cross-sectional view of the catheter assembly, illustrating an example shoulder, according to some embodiments;

FIG. 6 is a cross-sectional view of the catheter assembly, illustrating an example insert, according to some embodiments; and

FIG. 7 is a cross-sectional view of the catheter assembly, illustrating an example septum, according to some embodiments.

### DETAILED DESCRIPTION

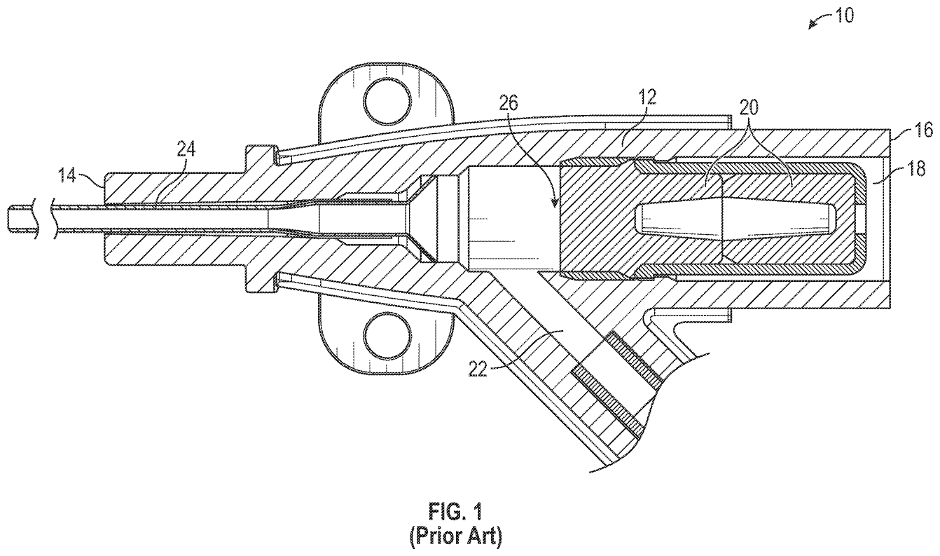

The present disclosure relates generally to vascular access devices, systems, and methods. More particularly, the present disclosure relates to a catheter assembly, as well as related devices and methods. Referring now to FIG. 1, a prior art catheter assembly 10 is illustrated. The prior art catheter assembly 10 includes a catheter adapter 12 with a distal end 14, a proximal end 16, and a lumen 18 extending through the distal end 14 and the proximal end 16. The prior art catheter assembly 10 includes a septum 20 spaced apart and proximal to a side port pathway 22. A catheter 24 extends distally from the distal end 14. The prior art catheter assembly includes a region 26 in which fluid may become stagnant despite flushing through the side port pathway 22. The region 26 includes a distal face of the septum 20 and an area within the lumen 18 adjacent thereto. Stagnant fluid within the region 26 may lead to an accumulation of bacteria within the prior art catheter assembly 10, which may result in infection or removal of the catheter 24 from the patient.

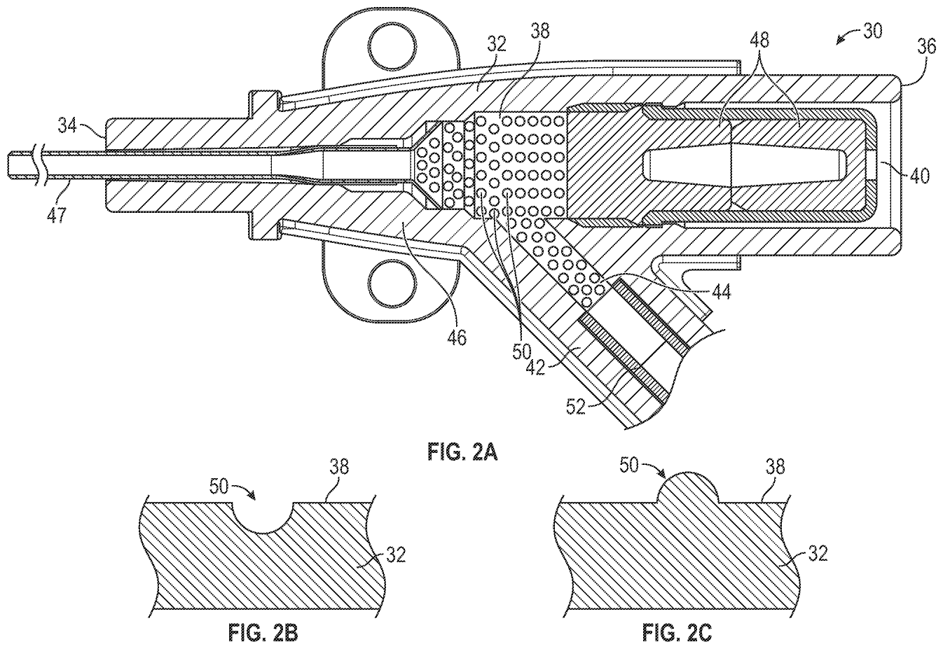

Referring now to FIGS. 2A-2C, a catheter assembly 30 is illustrated, according to some embodiments. In some embodiments, the catheter assembly 30 may include a catheter adapter 32, which may include a distal end 34, a proximal end 36, and an inner surface 38 extending through the distal end 34 and the proximal end 36. In some embodiments, the inner surface of the catheter adapter 32 may form a lumen 40. In some embodiments, the catheter adapter 32 may include a side port 42 forming a side port pathway 44 through a sidewall 46 of the catheter adapter 32 and in fluid communication with the lumen 40. In some embodiments, the sidewall 46 may include the inner surface 38.

In some embodiments, the catheter assembly 30 may include a catheter 47 extending distally from the distal end 34 of the catheter adapter 32. In some embodiments, the catheter 47 may include a peripheral intravenous catheter, a peripherally-inserted central catheter, or a midline catheter. In some embodiments, the catheter assembly 30 may include a septum 48, which may be disposed within the lumen 40 proximal to the side port pathway 44.

In some embodiments, a portion of the inner surface 38 proximal to the catheter 24 and distal to the septum 48 may include a textured surface. In some embodiments, the textured surface may be rough or uneven, which may increase a Reynolds number (Re) to promote turbulent flow. As illustrated in FIG. 2, in some embodiments, the textured surface may include multiple features 50, which may include depressions or dimples, as illustrated, for example, in FIG. 2B. In some embodiments, each of the features 50 may be dome shaped. In some embodiments, the features 50 may be arranged in a pattern, which may facilitate even disruption of fluid flow. For example, the features 50 may be disposed in one or more rows, which may be staggered from each other. In some embodiments, the features 50 may facilitate flushing of the catheter assembly 30 to avoid stagnant fluid within the catheter assembly 30. In further detail, the features 50 may induce turbulent flow within the catheter assembly 30. In some embodiments, the features 50 may include multiple protrusions or bumps, as illustrated, for example, in FIG. 2C, which may be arranged in the pattern and/or dome shaped.

In some embodiments, the textured surface may extend into the side port pathway 44. In some embodiments, an extension tube 52 may be integrated into the side port 42. In some embodiments, the textured surface may be proximate the extension tube 52, which may increase turbulence of fluid as the fluid enters the lumen 40 from the side port pathway 44. In some embodiments, the side port pathway 44 may be used for fluid infusion and/or blood collection. In some embodiments, a proximal end of the extension tube 52 may be coupled to a blood collection device.

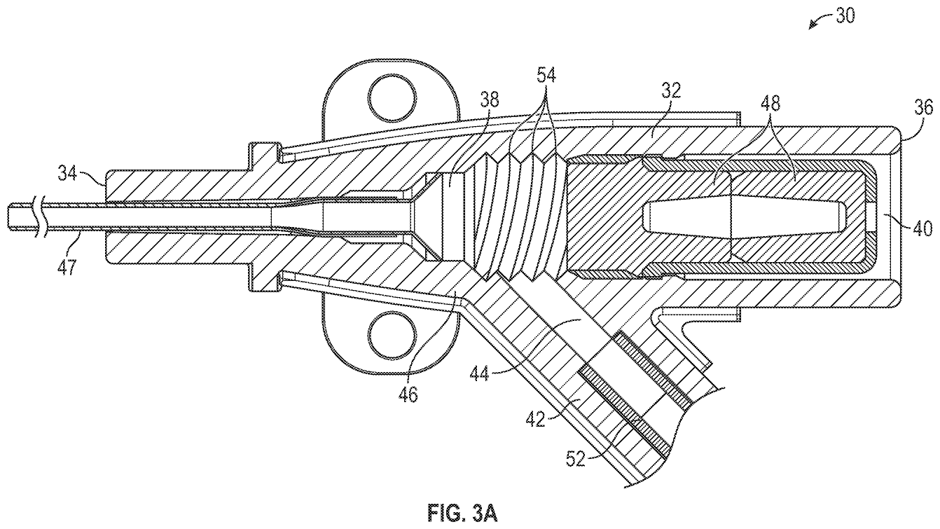

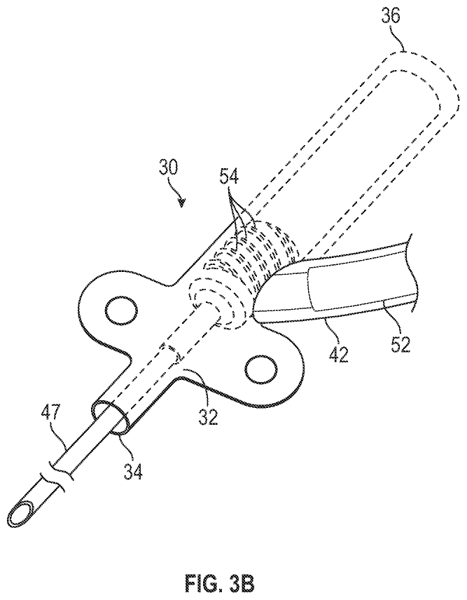

Referring now to FIGS. 3A-3B, the portion of the inner surface 38 proximal to the catheter 47 and distal to the septum 48 may include a channel configured to direct fluid flowing into the lumen 40 from the side port 42. In some embodiments, the channel may include a thread 54. In some embodiments, the thread 54 may wind helically around the inner surface 38, which may be generally cylindrical. In these embodiments, the portion of the inner surface 38 may be molded within a threaded core pin. In some embodiments, the thread 54 may intersect the side port pathway 44, which may facilitate travel of fluid from the side port pathway 44 into the thread 54.

In some embodiments, one or more of a pitch, angle, and shape of the thread 54 may be configured to direct fluid flow proximally. In some embodiments, the thread 54 may be angled toward the septum 48, as illustrated, for example, in FIGS. 3A-3B, which may direct fluid entering the lumen 40 from the side port 42 towards a region distal to the septum 48 where fluid may otherwise become stagnant or trapped.

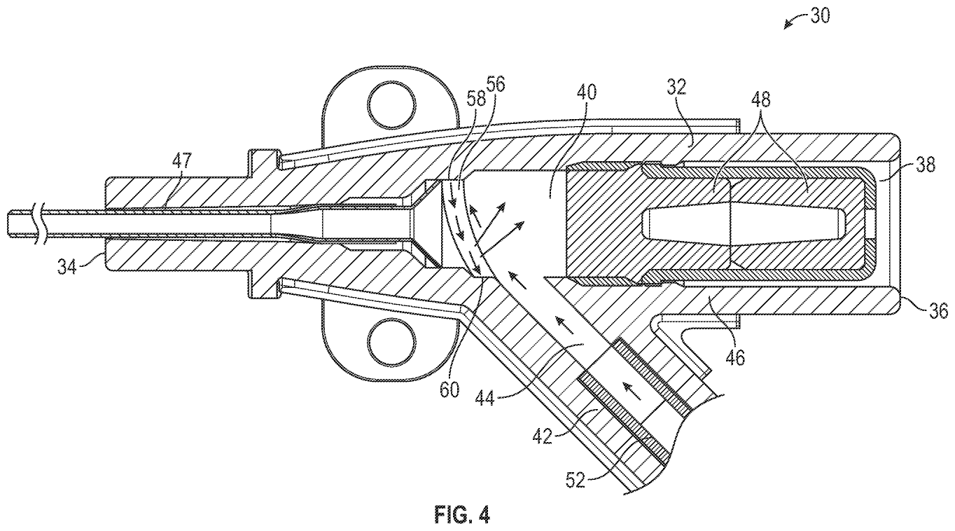

Referring now to FIG. 4, in some embodiments, the channel may include a groove 56, which may be disposed distal to the side port pathway 44. In some embodiments, fluid flowing into the lumen 40 from the side port 42 is configured to enter a first end 58 of the channel and flow towards a second end 60 of the channel. In some embodiments, the second end 60 of the channel may be disposed towards the side port 42 compared to the first end 58 of the channel. In some embodiments, the groove 56 may be angled in a distal to proximal direction, as illustrated, for example, in FIG. 4. In other embodiments, the groove 56 may be angled in a proximal to distal direction. In some embodiments, an angle of the groove 56 may increase fluid turbulence within the catheter assembly 30.

In some embodiments, the first end 58 of the channel and/or the second end 60 of the channel may be deeper than a portion of the channel aligned with a longitudinal axis of the catheter assembly 30, along which a probe may travel in a distal direction. In some embodiments, fluid flowing into the lumen 40 from the side port pathway 44 may flow across a width of the lumen 40 and repel off the first end 58. In some embodiments, the groove 56 may be configured to carry the fluid repelled off the first end 58 back across the width of the lumen 40 to the second end 60, where the fluid may be repelled again and toward the septum 48. In some embodiments, the groove 56 may create an unbalanced rotational effect on the fluid, which may facilitate greater turbulence and flushing. In some embodiments, the groove 56 may be positioned such that the probe does not catch or snag on the groove 56.

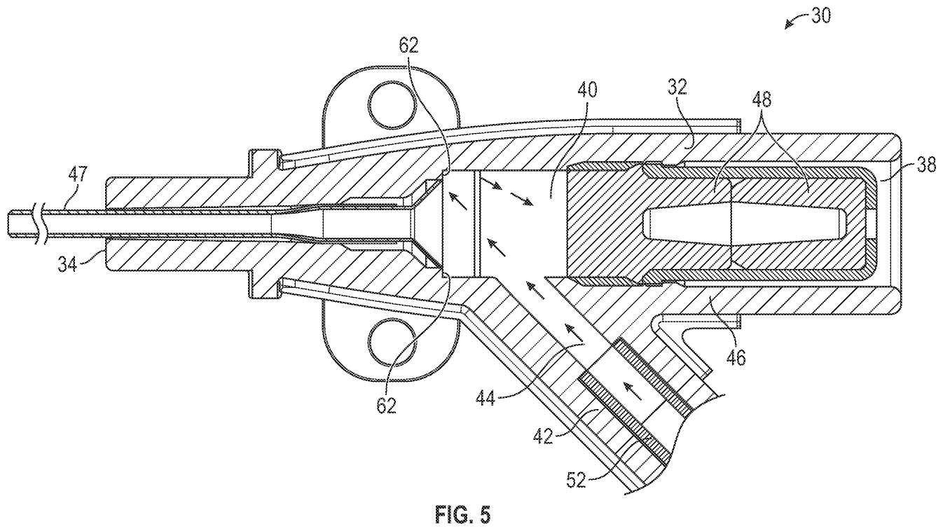

Referring now to FIG. 5, in some embodiments, the portion of the inner surface 38 proximal to the catheter 47 and distal to the septum 20 may include a shoulder 62, which may be annular. In some embodiments, the shoulder 62 may be disposed at approximately 90 degrees or between 60 degrees and 90 degrees. In some embodiments, the catheter assembly 30 may include a wedge 64, which may secure the catheter 47 within the distal end 34 of the catheter adapter 32, and the shoulder 62 may be proximate or contacting a proximal end 66 of the wedge 64. In some embodiments, fluid may flow into the lumen 40 from the side port pathway 44, and the shoulder 62 may be configured to divert the fluid proximally toward the septum 48.

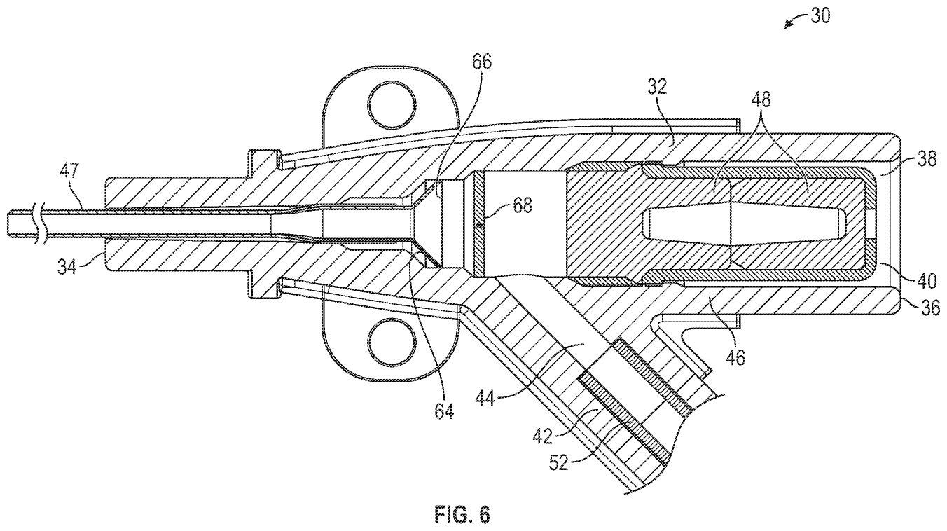

Referring now to FIG. 6, an insert 68 may be inserted distal to the side port pathway 44 and the septum 48. In some embodiments, the insert 68 may be proximal to the catheter 47 and/or a proximal end 66 of a wedge 64, which may be used to secure the catheter 47 within the catheter adapter 32. In some embodiments, the insert 68 many include a two-way valve, which may span the lumen 40 to provide a fluid seal. In some embodiments, the two-way valve may include a slit or leaflets, which may facilitate opening of the two-way valve at a predetermined pressure differential. In some embodiments, the two-way valve may be opened in response to infusion and/or blood draw via the catheter assembly 30. In some embodiments, the two-way valve may remain closed below the predetermined pressure differential and may open in response to a pressure differential exceeding the predetermined pressure differential. In some embodiments, flushing of the catheter assembly 30 may be improved due to all or substantially all of a space within the catheter adapter 32 proximal to the insert 68 and distal to the septum 48 being filled with fluid before the two-way valve opens. In some embodiments, the insert 68 may be elastomeric.

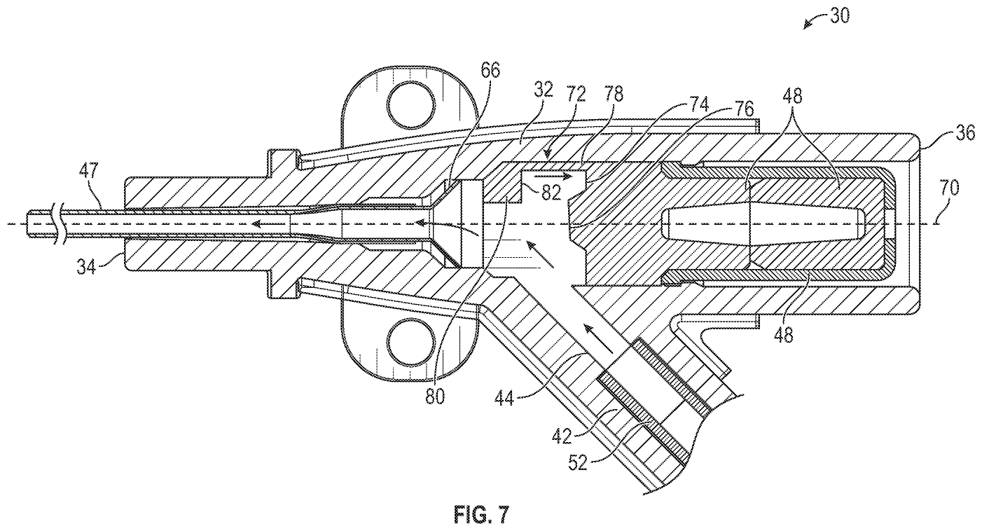

Referring now to FIG. 7, in some embodiments, the catheter assembly 30 may include a longitudinal axis 70. In some embodiments, a distal end 72 of the septum 48 may include an arm 78 extending distally and contacting the inner surface 38. In some embodiments, the septum 48 may include a distal face 74, which may be generally planar or which may include a protrusion 76. In some embodiments, the arm 78 may extend distally from the distal face 74. In some embodiments, the arm 78 may be on an opposite side of the lumen 40 as the side port 42 to facilitate fluid flow through the side port 42. In some embodiments, the arm 78 may decrease a volume of the lumen 40 in which fluid may otherwise become stagnant. In some embodiments, the arm 78 may extend distal to the side port pathway 44. In some embodiments, the arm 78 may extend to the wedge 64.

In some embodiments, a distal end of the arm 78 may include an extension 80 extending inwardly. In some embodiments, the extension 80 may extend inwardly toward the longitudinal axis 70. In some embodiments, the extension 80 may extend inwardly to or beyond the longitudinal axis 70. In some embodiments, the arm 78 and/or the extension 80 may be configured to direct fluid flow to the region. In some embodiments, the arm 78 and/or the extension 80 may be configured to direct blood proximally towards the distal face 74, which may facilitate flushing.

All examples and conditional language recited herein are intended for pedagogical objects to aid the reader in understanding the invention and the concepts contributed by the inventor to furthering the art and are to be construed as being without limitation to such specifically recited examples and conditions. Although embodiments of the present inventions have been described in detail, it should be understood that the various changes, substitutions, and alterations could be made hereto without departing from the spirit and scope of the invention.

What is claimed is:

1. A catheter assembly, comprising:
a catheter adapter, comprising a distal end, a proximal end, an inner surface extending through the distal end and the proximal end and forming a lumen, and a side port forming a side port pathway through a sidewall of the catheter adapter and in fluid communication with the lumen;
a catheter extending distally from the distal end of the catheter adapter;
a septum disposed within the lumen proximal to the side port pathway, wherein a portion of the inner surface proximal to the catheter and distal to the septum comprises a textured surface.

2. The catheter assembly of claim 1, wherein the textured surface is rough.

3. The catheter assembly of claim 1, wherein the textured surface comprises a plurality of depressions or protrusions.

4. The catheter assembly of claim 3, wherein each of the plurality of depressions or protrusions are dome shaped.

5. The catheter assembly of claim 4, wherein the plurality of depressions or protrusions is arranged in a pattern.

6. The catheter assembly of claim 1, wherein the textured surface extends into the side port pathway.

7. A catheter assembly, comprising:
a catheter adapter, comprising a distal end, a proximal end, an inner surface extending through the distal end and the proximal end and forming a lumen, and a side port forming a side port pathway through a sidewall of the catheter adapter and in fluid communication with the lumen;
a catheter extending distally from the distal end of the catheter adapter;
a septum disposed within the lumen proximal to the side port pathway, wherein a portion of the inner surface proximal to the catheter and distal to the septum comprises a channel configured to direct fluid flowing into the lumen from the side port.

8. The catheter assembly of claim 7, wherein the portion of the inner surface is molded within a threaded core pin, wherein the channel comprises a thread.

9. The catheter assembly of claim 8, wherein the channel intersects the side port pathway.

10. The catheter assembly of claim 7, wherein the channel is disposed distal to the side port pathway, wherein fluid flowing into the lumen from the side port is configured to enter a first end of the channel and flow towards a second end of the channel, wherein the second end of the channel is disposed towards the side port compared to the first end of the channel.

11. A catheter assembly, comprising:
a catheter adapter, comprising a distal end, a proximal end, an inner surface extending through the distal end and the proximal end and forming a lumen, and a side port forming a side port pathway through a sidewall of the catheter adapter and in fluid communication with the lumen;
a catheter extending distally from the distal end of the catheter adapter;
a septum disposed within the lumen proximal to the side port pathway, wherein a portion of the inner surface proximal to the catheter and distal to the septum comprises an annular shoulder.

12. The catheter assembly of claim 11, wherein the shoulder is disposed at a 90-degree angle.

13. The catheter assembly of claim 11, further comprising a wedge securing the catheter within the distal end of the catheter adapter, wherein the shoulder is proximate a proximal end of the wedge.

14. A catheter assembly, comprising:
a catheter adapter, comprising a body and a side port extending outwardly from the body, wherein the body comprises a distal end, a proximal end, and an inner surface extending through the distal end and the proximal end and forming a lumen, wherein the side port comprises a side port pathway extending through the side port and in fluid communication with the lumen;
a catheter extending distally from the distal end of the catheter adapter;
a septum disposed within the lumen; and
an insert disposed within the catheter adapter, wherein the insert extends across the lumen and selectively seals the lumen, wherein the insert comprises a two-way valve disposed distal to the side port pathway.

15. The catheter assembly of claim 14, wherein the insert comprises a slit or leaflets.

16. The catheter assembly of claim 14, wherein the insert is configured to remain closed unless a pressure differential exceeds a predetermined pressure differential.

17. A catheter assembly, comprising:
a catheter adapter, comprising a body and a side port extending outwardly from the body, wherein the body comprises a distal end, a proximal end, and an inner surface extending through the distal end and the proximal end and forming a lumen, wherein the side port comprises a side port pathway extending through the side port and in fluid communication with the lumen;
a catheter extending distally from the distal end of the catheter adapter; and
a septum disposed within the lumen, wherein a distal end of the septum comprises an arm extending distally and contacting the inner surface, wherein the arm is on an opposite side of the lumen as the side port.

18. The catheter assembly of claim 17, wherein a distal end of the arm comprises an extension extending inwardly.
